(19) United States
(12) Patent Application Publication
(10) Pub. No.: US 2013/0091939 A1
(43) Pub. Date: Apr. 18, 2013

(54) SENSORS FOR INTEGRATED MONITORING AND MITIGATION OF EROSION

(71) Applicants: Genda Chen, Rolla, MO (US); David Pommerenke, Rolla, MO (US); Zhi Zhou, Dalian (CN); Ying Huang, Rolla, MO (US); Andro Radchenko, Rolla, MO (US)

(72) Inventors: Genda Chen, Rolla, MO (US); David Pommerenke, Rolla, MO (US); Zhi Zhou, Dalian (CN); Ying Huang, Rolla, MO (US); Andro Radchenko, Rolla, MO (US)

(21) Appl. No.: 13/693,139

(22) Filed: Dec. 4, 2012

Related U.S. Application Data
(63) Continuation-in-part of application No. 13/104,682, filed on May 10, 2011.

(51) Int. Cl. G01N 33/24

(52) U.S. Cl. CPC G01N 33/24
USPC 73/86

(57) ABSTRACT

Methods and systems for measuring erosion. Systems of various embodiments include a sensor adapted to be placed where earthen material is expected to move and to sense a condition related to that movement (for instance, the position of the sensor). The sensor includes a receiver for receiving a wireless signal (be it acoustic, magneto-inductive, etc.) from another sensor which conveys an identifier for the second sensor. The first sensor also includes a signal generator that generates a second (possibly wireless) signal conveying that identifier and its own identifier. Systems of some embodiments include a second receiver placed outside of the region. If desired, the sensor can determine the signal strengths of the signals that they receive from the other sensor and can convey an indication of the received signal strengths. Furthermore, some sensors include accelerometers, roll sensors, tilt sensors, yaw sensors, magnetometers, etc.

### CROSS REFERENCE TO RELATED APPLICATIONS

This application is a continuation in part of U.S. patent application Ser. No. 13/104,682 entitled “Sensors For Integrated Monitoring and Mitigation of Scour,” filed on May 10, 2011 by Dr. Genda Chen et al., the entirety of which is incorporated herein as if set forth in full and which claims priority to U.S. Provisional Patent Application No. 61/333,046, filed on May 10, 2010, entitled “Sensors For Integrated Monitoring And Mitigation Of Scour,” by Dr. Genda Chen et al. the entirety of which is also incorporated herein as if set forth in full.

### BACKGROUND

Scour is a process in which a fluid erodes material supporting a structure away from that structure. When scour occurs near a bridge, the associated erosion can cause that bridge to collapse. More particularly, bridge scour is an erosion process in which the current of a river erodes soil deposits around the foundation (piers, abutments, etc.) of a river-crossing bridge. Of course, scour can occur in many bodies of water and near other structures. For instance, bodies of salt water can give rise to scour around piers, walls, levees, etc. More specifically, with bridge scour, portions of the bridge foundation interact with the flow of the river thereby creating eddies and other phenomenon (for instance localized impingement of high speed water on portions of the riverbed) which lead to the erosion. Bridge scour (as well as other forms of scour) is therefore often characterized by the formation of scour holes, dunes, etc. around the bridge foundation.

Scour is a world-wide issue of growing concern. For instance, in the United States, scour-related erosion causes more bridge collapses than any other condition. As of 1997, more than 10,000 bridges out of the 460,000 over-water bridges in the United States were scour critical and 132,000 were scour susceptible. By 2005, however, approximately 26,000 bridges had become scour critical and more than 190,000 bridges had become scour susceptible. With the recent spate of floods, it is likely that even more bridges have become scour critical, potentially resulting in failure of some of these bridges.

### SUMMARY

The following presents a simplified summary in order to provide a basic understanding of some aspects of the disclosed subject matter. This summary is not an extensive overview of the disclosed subject matter, and is not intended to identify key/critical elements or to delineate the scope of such subject matter. A purpose of the summary is to present some concepts in a simplified form as a prelude to the more detailed disclosure that is presented later.

Generally, this document describes embodiments of sensors, systems, and related methods which can help tackle challenges associated with monitoring and mitigating erosion in general and scour more particularly. More specifically, one embodiment provides a system for measuring the erosion around bridges caused by scour. These systems can include sensors designed to mimic naturally occurring rocks. This configuration of the sensors enables users to drop the sensors into a river thereby mitigating the scour in some cases.

Generally, embodiments provide systems which work on the following principals (among others):
Passive systems in which the sensors generate a magnetic field or alter the surrounding magnetic field (via a magnet or piece of magnetic material) which is used to detect individual sensors or groups thereof.
Passive systems in which the sensors scatter a magnetic field with a resonator using magneto-inductive communication thereby allowing the detection of individual sensors or groups thereof.
Active systems in which the sensors have no internal power source. Rather, they receive power through magneto-inductive power coupling or power harvesting to power an active circuit that turns on/off at select times thereby enabling selective transmission of scour related data sensed by the sensors.
Active systems in which the sensors contain an internal power source (for instance, they contain a battery) or receive power from an external magnetic field. Sensors of the current embodiment sense scour related conditions and transmit data regarding the same through magneto-inductive, ultrasonic, or other suitable forms of wireless communication. Some embodiments provide sensors containing a timer so that they can transmit at select times (for instance, every hour). The transmitted data can include data regarding the battery status, an identification of the sensor, the orientation of the sensor, and/or other scour related data. In some embodiments the sensors contain a magnet (and, optionally, a housing for the magnet) so that the positions of the sensors can be magnetically measured as scour moves the sensors about on the riverbed. In other embodiments, the sensors include active components (in addition to, or in the alternative to, passive magnets) which can detect scour-related condition(s) and can cause information related to scour to be transmitted to a receiver. These active sensors can also be configured to mimic naturally occurring rocks.

Embodiments also provide integrated scour monitoring and mitigation systems. These systems can measure the motion of the sensors as the sensors move about under the influence of liquid in which they are submerged. In some embodiments, the motion of individual sensors is measured whereas in other embodiments the motion of a group of sensors is measured. Whether individual sensors are tracked, or groups of sensors are tracked, the mobility of the sensors can indicate the scour susceptibility/criticality of various monitored structures.

Systems of some embodiments can include a group of such sensors (and other types of sensors if desired), each with an embedded magnetic device in wireless communication with a measurement instrument (such as a magnetometer). These sensors can also possess densities sufficient to cause them to sink in, yet be moved by, flowing water in a manner similar to naturally occurring rocks (or other filler material). Systems of such embodiments can be used to monitor, prevent, and mitigate riverbed scour-related conditions near bridge foundations. In some embodiments, a user places the sensors near the foundation of a bridge before, after, or even during a scour event. When floods or other scour-inducing events occur, the river current typically moves (or at least re-orients) some of the sensors. As a result of the movement and/or reorientation of the sensors, the three dimensional magnetic field caused by the group of sensors measurably changes. Hence one can observe changes in the magnetic fields during flood conditions, as they indicate movement of sensors, or one can, using many field measurement points reconstruct the location of sensors, without waiting for changes. Changes in the location or orientation of the sensors can be related to characteristics of the scour associated with the bridge foundation. For instance, the as-sensed changes in the magnetic field can be related to the time-varying depth, width, and locations of voids and accumulations of material in or on the riverbed.

Some embodiments provide systems which include a sensor and a signal generator with a combined density equal to or greater than that of water. Optionally, the sensor can be a magnet, resonator, or accelerometer. Moreover, the sensors can be adapted to be placed in regions potentially subject to scour and to sense scour-related conditions. The signal generator of some sensors generates a wireless signal conveying data regarding the as-sensed scour-related. In some embodiments the sensor is the signal generator while a receiver for the wireless signal can include an antenna, a magnetometer, or an ultrasonic sensor. In some embodiments, the housing is conic and the magnetic object is offset from the center of gravity of the coupled sensor, signal generator and housing.

In methods implemented in conjunction with various embodiments, sensors can be placed near existing bridges shortly before (for instance, about one day before) a predicted flood or other scour-inducing event. Since sensors of various embodiments can be dropped into place (or otherwise positioned) and their movements and orientations tracked, such techniques can allow real-time and cost-effective monitoring of scour. Thus, systems of various embodiments can facilitate evaluation of the scour-related condition of bridge foundations and can enable damage reduction, mitigation, prevention, etc. Sensors of various embodiment and/or other types of scour sensors can be applied to many structures (for instance, sea-crossing bridges, levees, pipes undersea cables, etc.) with results similar to those disclosed above. Should scour be detected, sensors and other filler material (artificial objects, naturally occurring rocks, etc.) can be placed near a scour critical (or other) structure to stabilize it based on real-time, reliable, and robust data obtained from various sensors.

Yet other embodiments provide methods of monitoring and/or mitigating scour. In some of these methods, at least one sensor with a density about equal to or greater than water (for instance densities between about 1.2 g/cm̂3 and about 5.3. g/cm̂3) is placed in water at a location where the water is expected to flow and (potentially) cause scour. The sensor includes an object which alters the magnetic field in its vicinity in response to a change in a scour-related condition at about the location of the sensor. Additionally, such methods include allowing a scour-related change to occur at or near the sensor and allowing the sensor to cause a wireless signal to propagate through the water to convey data regarding the scour-related condition.

In some methods the sensor can include a signal generator in communication with the sensor to cause (or transmit) the wireless signal. The signal generator can be a passive magnet, an actively powered magnet, a magnetic resonator, an accelerometer or a combination thereof with, or without, other types of instruments. If desired, the object can be the signal generator. Moreover, in methods of some embodiments, the wireless signal is received and the data conveyed thereby is correlated to determine the scour-related condition.

Some embodiments provide methods for measuring erosion. For instance, some of these methods include placing a sensor (having an associated sensor identifier) in a region through which earthen material is expected to move. These methods also include sensing a condition related to the movement of the earthen material with the sensor and receiving a first wireless signal conveying a sensor identifier associated with a second sensor. Moreover, these methods also include generating a second signal conveying the first sensor identifier and the second sensor identifier and locating the first sensor using the second signal. In some cases, the second signal is wireless too and it can be used to locate the second sensor. Furthermore, locating of the second sensor can include using an identifier/distance pair associated with the second and first sensors which was conveyed by the second signal. Note that sensors of embodiments can “sense” their own location by producing a signal (magnetic, magneto-inductive, acoustic, etc. which identifies their location when triangulated or otherwise determined).

Still other embodiments provide systems for measuring erosion wherein the systems each include at least a first sensor. The first sensor is adapted to be placed in a region through which earthen material is expected to move as the erosion occurs and to sense a condition related to that movement. For instance, the position of the sensor can be that condition. In addition, the sensor has an identifier associated with it. In operation, the sensor receives a first wireless signal from another sensor wherein that signal conveys a second identifier associated with the other sensor. The first sensor also includes a signal generator that generates a second signal conveying the identifiers of both sensors (if available).

Moreover, in some embodiments, the second signal is also a wireless signal and the system includes the second sensor. The first sensor can be mounted on a structure with a bracket or it can be so dense (or heavy) that it is unlikely to be moved during erosion events. If desired, the sensor can determine the signal strength of the signal that it receives from the other sensor and can convey an indication of that signal strength in the first wireless signal. Furthermore, some sensors include accelerometers, roll sensors, tilt sensors, yaw sensors, magnetometers, etc. and the first wireless signal can be an acoustic or magneto-inductive signal.

Other embodiments provide methods for measuring erosion. Such methods include placing a first sensor (having a sensor identifier) in a region through which earthen material is expected to move. A condition related to the movement of the earthen material is detected using the first sensor. For instance, the sensed condition can be the location of the first sensor. These methods also include receiving a first wireless signal (using the first sensor) conveying a sensor identification associated with a second sensor and generating a second signal conveying the first and the second identifiers.

If desired, the second signal can also be wireless. The signal strength of the first signal as it is received can be determined and (if desired) the second signal can also convey an indication of that received signal strength. Furthermore, some methods include sensing the acceleration of the first sensor.

To the accomplishment of the foregoing and related ends, certain illustrative aspects are described herein in connection with the following description and the associated figures. These aspects are indicative of various ways in which the disclosed subject matter may be practiced, all of which are intended to be within the scope of the disclosed subject matter. Other advantages and novel and non-obvious features may become apparent from the following detailed description when considered in conjunction with the figures.

### BRIEF DESCRIPTION OF THE FIGURES

The detailed description is written with reference to accompanying figures. In the figures, the left-most digit(s) of a reference number identifies the figure in which the reference number first appears. The use of the same reference numbers in different figures indicates similar or identical items.

FIG. 1 illustrates an integrated scour measurement and mitigation system.

FIG. 2 illustrates causes of bridge collapse.

FIG. 3 illustrates growth in the number of scour susceptible bridges.

FIG. 4 illustrates scour associated with a bridge.

FIG. 5 illustrates a comparison of various scour measurement systems.

FIG. 6 illustrates a passive scour measurement and mitigation system.

FIGS. 7A-7D illustrate position response functions of some passive scour sensors.

FIG. 8 illustrates signal intensity-distance curves associated with passive scour sensors made of various materials.

FIG. 9 illustrates an active scour measurement and mitigation system.

FIG. 10 illustrates an erosion measurement system.

FIG. 11 illustrates another erosion measurement system.

FIG. 12 illustrates yet another erosion measurement system.

FIG. 13 illustrates still another erosion measurement system.

FIG. 14 schematically illustrates an erosion sensor.

FIG. 15 illustrates a block diagram of an erosion measurement system.

FIG. 16 illustrates a flowchart of a method of measuring erosion.

### DETAILED DESCRIPTION

This document discloses techniques and technologies for monitoring erosion related conditions. More particularly, this document discloses techniques and technologies for integrated monitoring and mitigation of hydraulic scour associated with bridge foundations and other structures (for instance levees).

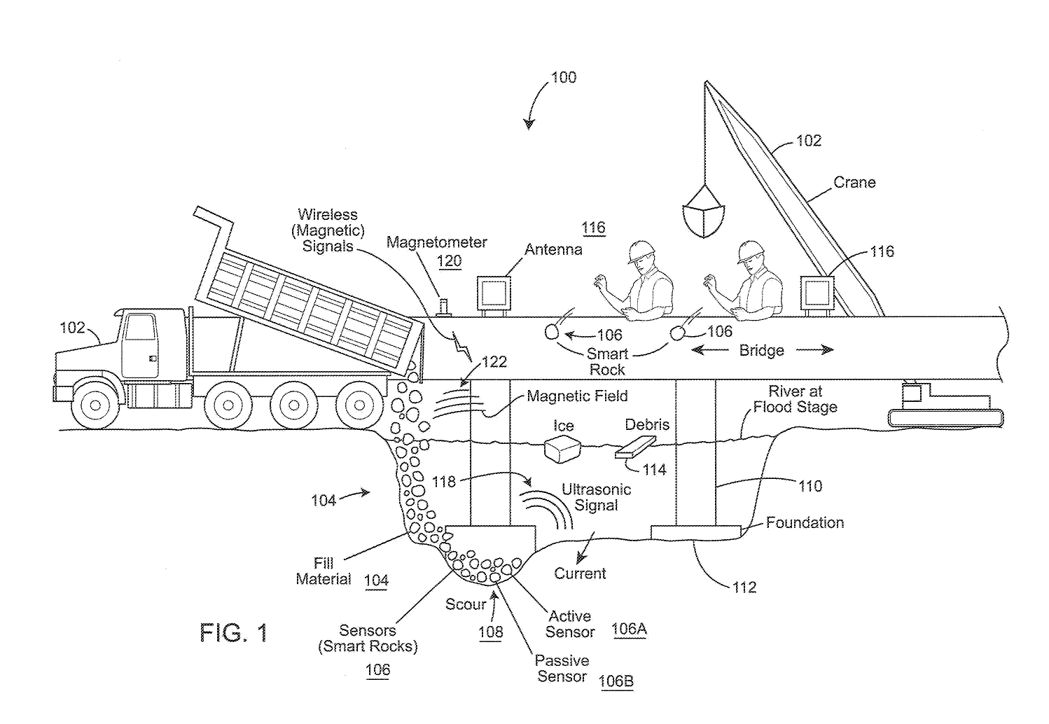

FIG. 1 illustrates an integrated scour measurement and mitigation system. The system 100 of the current embodiment provides integrated scour monitoring and mitigation. As illustrated by FIG. 1, a vehicle 102 (for instance, a truck, barge, crane, etc.) can deposit a volume of filler material 104 including various sensors 106 (for instance, active and passive sensors 106A and 106B respectively) in place near a potential or existing region 108 of scour. In the situation illustrated by FIG. 1, one pier 110 and footing 112 of a bridge foundation has experienced some scour during an ongoing flood. Since the flood waters might be carrying various pieces of debris 114, ice, and the like, it is difficult, if not impossible, to apply previously available scour monitoring technologies to the bridge. Nonetheless, the vehicle 102 is able to position the filler material 104 and sensors 106 (active and passive sensors 106A and 106B in this scenario) in the region 108 undergoing scour.

For the illustrated situation, scour has occurred close enough to shore that a truck can deliver the filler material 104 and sensors 106 to the scour site. If the scour had occurred further from shore or at some other location inaccessible to a truck, then a crane, barge, or other device could be used to deliver the filler material 104 and sensors 106 to the site. Moreover, users can drop additional sensors 106 into the water at the region 108 of interest as is shown in FIG. 1 where the users are dropping the sensors 106 into the water on the upstream side of the bridge. Thus, should the sensors 106 be misplaced, the current is more likely than not to wash the sensors 106 into the scour site.

These users have also deployed a pair of antennas 116 to receive wireless magnetic signals 118 from the sensors 106. In addition, in this case, the users have deployed a magnetometer 120 to sense the magnetic field 122 associated with the passive sensors 106B. As disclosed further herein, though, other communication methods can be employed. For instance, the sensors 106 can use ultrasonic communication, can back scatter RF signals using resonators at the same frequency or at the frequencies of the resonators (positioned in various receiving devices located on shore or elsewhere). From the data gathered by the antennas 116 and the magnetometer 120, the users can derive information related to the location and dimensions of various scour-related voids and formations near the bridge.

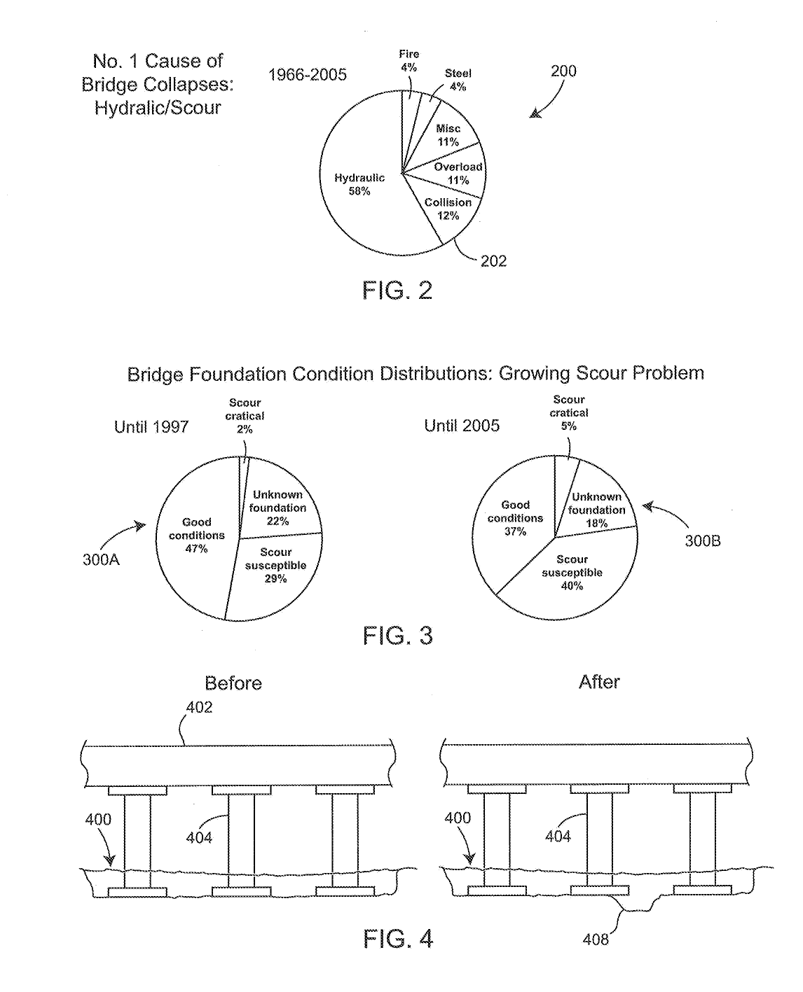

Bridge collapses due to scour often occur rapidly, sometimes within hours or days from the onset of scour critical conditions. FIG. 2 shows the various causes 200 of bridge collapses between 1966 and 2005. See Briaud, J. L., and Hunt, B. E., “Bridge Scour & the Structural Engineer,” Structures Magazine, December 2006, pp. 58-61. Hydraulic scour (at 58% as shown in FIG. 2) is the greatest cause 200 for bridge collapses. Bridge scour as a growing issue is clearly seen by comparing the 2005 and 1997 pie-charts 300A and 300B of FIG. 3. See Abutments,” NCHRP Report 396, Transportation Research Board, National Research Council, National Academy Press, Washington, D.C., 1997, p. 109 and see Hunt, B. E., “Practices for Monitoring Scour Critical Bridge,” NCHRP Report 205, Transportation Research Board, 2005, p. 8. Some of the consequences of bridge scour are shown in FIG. 4 in which a flood has eroded the riverbed 400 near a bridge 402 foundation 404 to cause a region of scour 408. Present methodologies with portable and fixed instruments, such as 1) inspection by divers, 2) probing rods, 3) ground penetrating radars (GPR), 4) boats, 5) sonar systems, 6) float-out devices, 7) magnetic sliding collars, 8) optical sensor systems, and 9) global positioning systems can hardly be applied when strong currents and/or floating debris 114 and/or ice exist in a river. These and other conditions therefore complicate proper application of such heretofore available techniques.

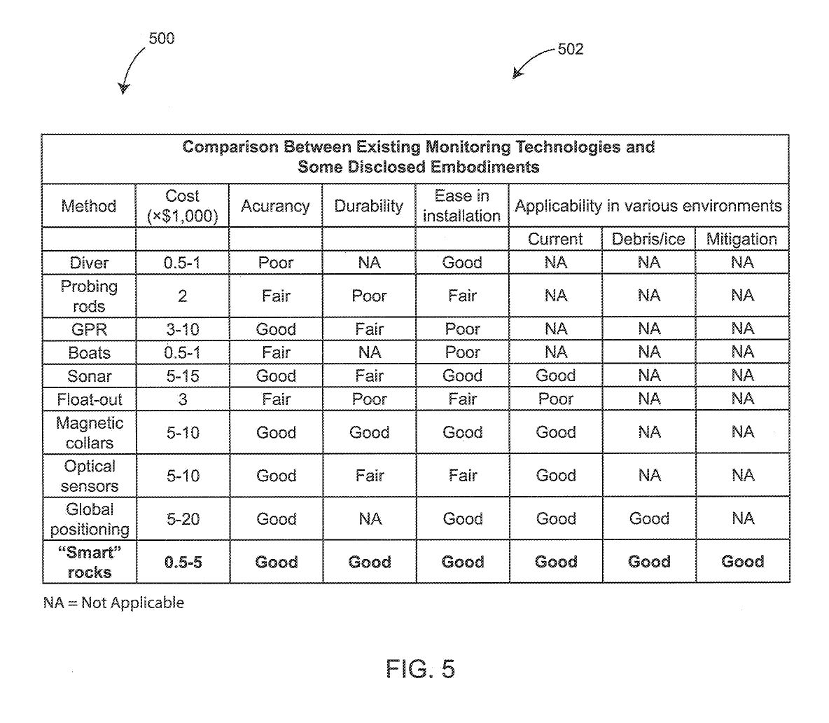

Furthermore, while a number of approaches exist for measuring scour-related conditions, previously available approaches suffer from certain disadvantages. FIG. 5 illustrates certain considerations with respect to monitoring and mitigating scour-related conditions with various types of systems including cost, accuracy, durability, ease of installation, applicability under certain condition, etc. See Federal Highway Administration (FHWA) and National Highway Institute (NHI), “Bridge Scour and Stream Instability Countermeasures: Experience, Selection, and Design Guidance,” Second Edition, Publication No. FHWA NHI 01-003, Hydraulic Engineering Circular No. 23, March 2001. See also Iowa Highway Research Board (IHRB), “An Illustrated Guide for Monitoring and Protecting Bridge Waterways against Scour,” Final Report No. 449, Project TR-515, March 2006. FIG. 5 also shows that some approaches or methods 500 can be applied with various results 502. However, previously available technologies can only be applied before and after scour-inducing events and cannot be used to mitigate scour-related conditions (unlike at least some of the sensors 106 disclosed herein).

### Sensors

Embodiments disclosed herein can be successfully applied to structures before, after, and even during scour-inducing events. Thus, knowledge of scour-related conditions can be obtained during all periods pertinent to the monitored structures. More particularly, scour sensors can be placed in regions 108 where scour is likely even during a time when scour susceptible and scour critical conditions might arise (for instance, during a flood). Various embodiments disclosed herein can therefore provide interested users with real-time information pertinent to understanding and evaluating changes that occur during such events (in addition to before-and-after comparisons of scour-related conditions).

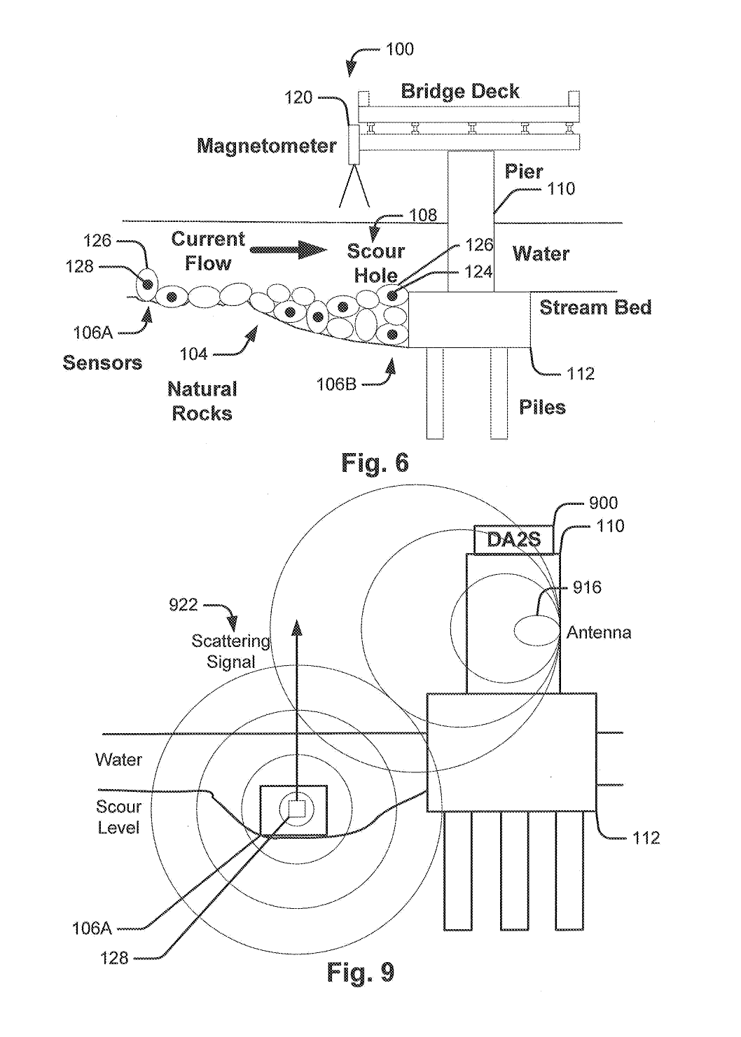

More specifically, systems of some embodiments include rock-like objects or concrete blocks with 1) embedded passive or active electronics, 2) a physically separate monitoring station or receiver, and 3) a wireless communication link there between so that related parameters (the locations of the sensors, the density of a group of sensors, their proximity to neighboring sensors, the acoustic noise or vibration level in the river, etc.) can be determined under strong flooding (or other) conditions. The information derived there from can enable scour evaluations and mitigation in real time. Embodiments disclosed herein include passive sensor embodiments and active sensor embodiments as illustrated by FIGS. 6-9 and elsewhere herein.

Real-Time Scour Mounitoring with Passive Sensors Group Dispersion Methods

Some passive sensor 106B embodiments involve creating a constant magnetic field 122 about each of (or some of) a group of passive sensors 106B. As disclosed further herein, the constant (with respect to the sensors 106) magnetic fields 122 can be used for locating them as a group (passive sensor group dispersion) or individually. Magnetometers 120 can be used to measure the intensity of the combined magnetic fields 122 from the Earth, the passive sensors 106B, and the ferromagnetic parts around a bridge foundation or other structures.

In some passive embodiments, each passive sensor 106B includes a magnet 124 embedded in a housing 126 (see FIG. 6). These passive sensors 106B can be configured to have a density similar to that of naturally occurring rocks (or other filler material 104) used to mitigate scour. For instance, some passive sensors 106B (the embedded magnets and the housings combined) possess densities between about 1.2 g/cm̂3 and about 5.3 g/cm̂3 although other densities are also within the scope of the disclosure. Moreover, some housings can be shaped, dimensioned, etc. to mimic naturally occurring rocks in the locale of interest so that the passive sensors 106B move in response to flowing water in a manner similar to those naturally occurring rocks. Some embodiments provide more or less spherical sensors 106 with diameters of about 50 cm although sensors 106 of other dimensions (smaller and larger) and/or shapes are within the scope of the disclosure. For instance, in some embodiments, the sensors 106 are cone shaped with a center of gravity positioned to cause them to settle standing on their base. Thus, should scour remove the material underneath such sensors 106 they will tip over causing a detectable change in their orientation.

Various methods can be used to increase the magnetic fields 122 of passive sensors 106B. For instance, instead of using one passive sensor 106B, a group of passive sensors 106B can be placed near a bridge. Since the group will function like a large multi-pole magnet 124 (with the resulting magnetic field 122 reflecting contributions from each of the individual passive sensors 106B), the resulting magnetic field 122 can be used to detect the location of the group of passive sensors 106B. In addition, or in the alternative, the magnets 124 of a group of passive sensors 106B can be allowed to align themselves with the surrounding magnetic field by fixing the magnets 124 after the passive sensors 106B have been put in place. Not only might the alignment increase the magnetic field but it might also create a magnetic field which reflects the uniform orientation of the magnets.

Another way to align these magnets 124 is to insert the magnets 124 into holes in the sensor housings. The holes can be shaped to correspond to the shapes of the magnets 124 while leaving gaps between the magnets 124 and the housings 126. These gaps can be filled with an epoxy or some other material that will eventually set within a selected time (such as 10-30 minutes) thereby fixing the magnets 124 in the housing. The resulting passive sensors 106B can be sealed and placed in the water at desired locations while the gap-filler material begins setting as shown in FIG. 6. It is noted here that, the gap-filler material can be selected so that by the time it sets, the magnets 124 in the as-placed sensors 106 have re-oriented themselves to align themselves as follows. In one embodiment, the centers of gravity of the magnets 124 are offset from the geometric center of the sensors 106 such that when the sensors 106 settle, the sensors 106 rotate with their heaviest portion positioned toward the bottoms of the sensors 106. Thus, if all of the magnets 124 of the sensors 106 have similar magnetic orientations (relative to the axes defined by the offsets of the geometric centers and center of gravities) then all of the sensors 106 will be magnetically aligned once they have settled and the gap-filler material sets.

In another embodiment, the magnets 124 align themselves with the surrounding magnetic field (often the Earth's magnetic field) as follows. In the current embodiment, the magnets 126 do not have an offset between their centers of gravity of the magnets 124 and the geometric centers of the sensors 106. Instead, the magnets 124 remain free to rotate in accordance with the surrounding magnetic field until the gap-filler material sets. Thus, once the magnets 126 are inserted into the sensors 106 and the sensors 106 settle, the magnets 126 rotate to align themselves with the surrounding magnetic field. Since the surrounding magnetic field will generally be that of the Earth, the individual magnets 126 will align with the Earth's magnetic field. Further, since all of the magnets 126 are aligned with a common reference (the Earth's magnetic field), they can all be aligned with each other if desired. Such sensors 106 can find application in situations where the sensors 106 near a particular structure are, or will be, dispersed from one another.

In the alternative, or in addition, steel blocks can be embedded into some passive sensors 106B to concentrate or focus pre-existing magnetic fields in their vicinity. Since the steel blocks cause no magnetic field of their own, it is likely that such sensors 106 can be used without orienting the steel blocks. Thus, in various embodiments, the magnets 124 of the sensors 106 can be aligned with each other thereby providing a magnetic field 122 reflecting that uniform alignment and which is stronger than it would be were the sensors 106 were not aligned.

Sensors of some embodiments include magnets 124 which are configured to always point up to facilitate locating such sensors. More particularly, since the dipole moment orientations of the magnets are known (or can be measured) prior to placing such sensors in an area of interest, these sensors can be more readily located than those in which the magnet might rotate with the sensor. To create a sensor 124 of the current embodiment, the magnet can be allowed to rotate within an asymmetric sensor body so that the south pole always point up.

Whether the passive sensors 106B are aligned or not, an instrument such as a magnetometer 120 can measure the resulting magnetic field 122 produced by the in-situ sensors 106 and changes to the same. For instance, when three-dimensional scour-related data is desired, several (for instance, three or more) magnetometers 120 can be used to enable real-time evaluation of bridge scour (in terms of the maximum depths of scour-induced holes as well as other riverbed changes) by an evaluation of the positions of the sensors. In some cases, this evaluation process can use an inverse transformation to identify the presence and location of a group of sensors 106 from the measured magnetic field data. Thus, the passive sensors 106B sense (by their presence in the resulting riverbed formations) the scour related erosion of the riverbed. The tracking of the passive sensors 106B can be performed continuously (providing real-time scour information if desired) or on a selected schedule. Moreover, the locations of the passive sensors 106B as a group can be tracked to provide scour-related information.

At times it might be found desirable to add passive sensors 106B to a particular location. For instance, should some of the passive sensors 106B move away from the bridge, more sensors 106 can be added to the site. Indeed, in some cases, it might be useful to have about 10% to about 30% of the filler material 104 at a particular location be sensors 106 as shown in FIG. 6. Furthermore, because the passive sensors 106B can be configured to mimic naturally occurring rocks (or other filler materials 104) the placement of passive sensors 106B at the site can mitigate scour conditions.

Even so, during a scour-inducing event, the passive sensors 106B (and other objects and materials) in the water will likely be washed away or re-oriented. As a result, the combined magnetic field 122 (and/or the topology thereof) of the passive sensors 106B group will change in a corresponding fashion. Indeed, whereas a group of deployed sensors 106 will have an initial magnetic field 122 reflecting their originally deployed orientation (in line with the surrounding magnetic field, having an orientation of its internal DC magnetic field 122 parallel to the gravity-oriented magnetic field 122, etc.), a group of sensors 106 disturbed by a scour event will likely exhibit a changed magnetic field 122. In many cases, the signature of the magnetic field 122 of the passive sensors 106B will be randomized as compared to the original signature. As noted elsewhere herein, these changes can be measured. Thus, the data obtained from such sensors 106 can signify the onset and level, or degree, of bridge scour. In other words, the randomization of the magnetic field alone indicates that erosion has occurred. Further measurement and analysis of the magnetic field can determine the extent of that erosion.

### Experimental Passive Sensor Systems

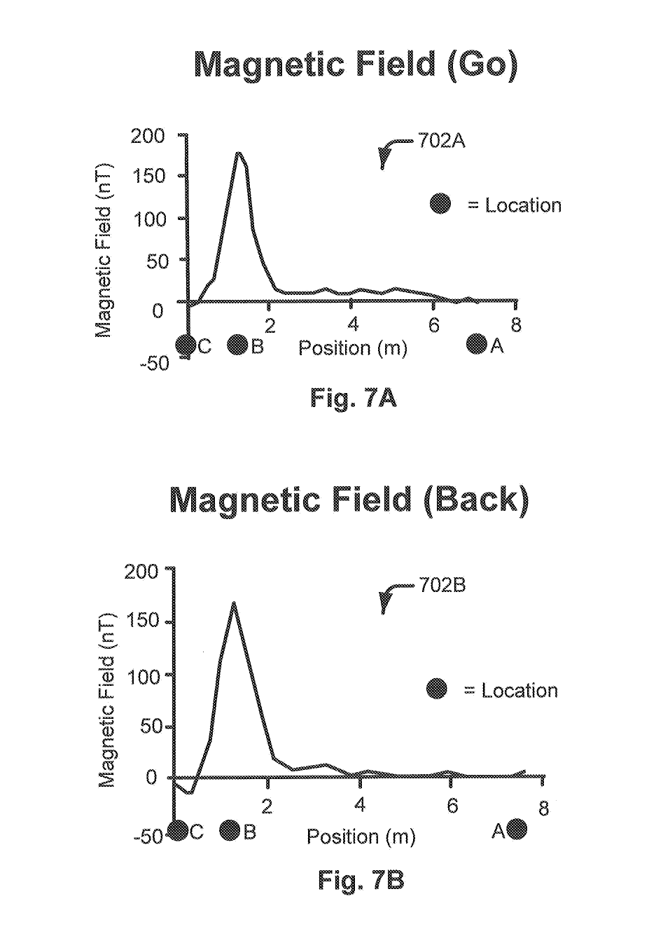

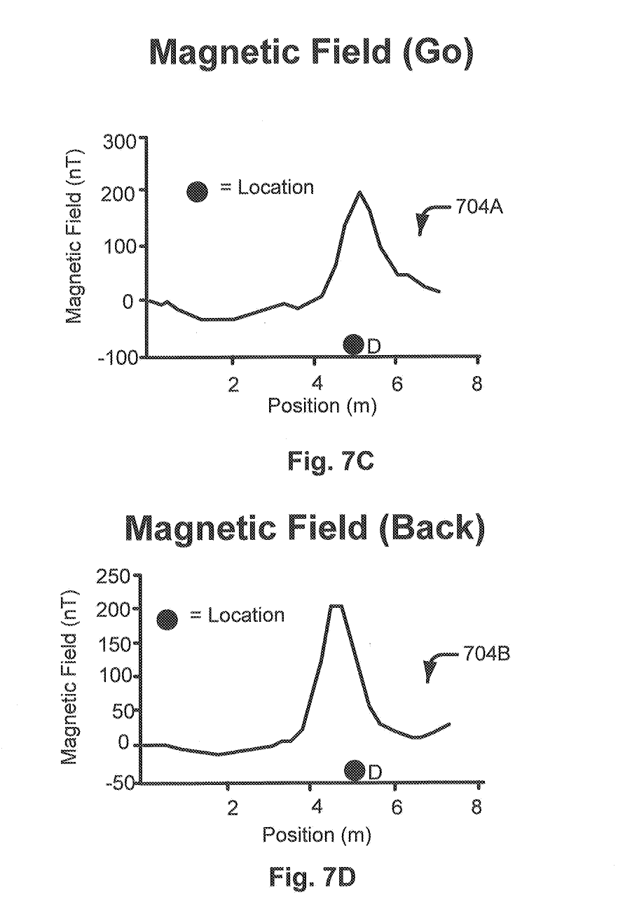

Initial tests of a passive system 100 were recently conducted at the Missouri University of Science and Technology (hereinafter “Missouri S&T). The experimental passive system 100 included magnets emulating the sensing unit of passive sensors 106B. In these tests, three groups of magnetic objects were tested at Missouri S&T and include a 6 cm magnet cube, 0.75 cm×15 cm×75 cm steel plates, and 15 cm-long #8 steel bars. Each of these magnetic objects was pulled in one direction and its position was detected with a model number G858 Geometrics magnetometer 120 (available from the Oyo Corporation USA in San Jose, Calif.). Plots 702A and 702B (of FIGS. 7A and 7C respectively) are representative of the data gathered. Note that the variation in the value between the two functions to the right of Point B was attributed to the effect of some small extraneous hand-held metallic objects being moved about near the experimental system.

FIGS. 7C and 7D also present the plots 704a and 704B of the intensity of a magnetic field as measured by the magnetometer as it was moved from a Point A to a Point B (7.6 m apart) and then returned to Point A while a 15 cm long #8 steel reinforcing bar was left at a fixed location D. As shown by FIG. 7 the plots 704 of the intensity of the magnetic field 122 drops with increasing distance between the magnetometer and the reinforcing bar. However, the position response function (the plots 704) of FIG. 7 clearly indicates the location of the bar with a spatial resolution of less than 1.0 m between two identical bars. The variation between the two plots 704 of the intensity arise primarily from hysteresis. The sensitivity of the magnetometer 120 to these objects demonstrates the ability of the experimental system 100 to measure the movement of metallic objects in a particular volume of interest.

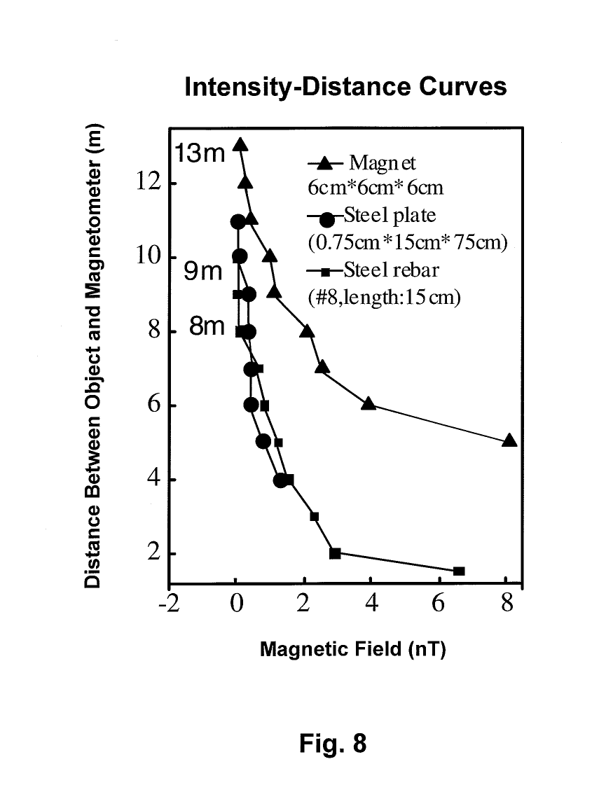

FIG. 8 presents plots 802 of the magnetic field intensity as a function of distance from several metallic items as measured by the experimental system 100. The magnetic objects included a magnet, a steel plate, and a piece of steel reinforcing bar. In the experimental system 100, a practical measurement distance happens to be between about 8 and about 13 m depending on the type of metallic objects in the region 108 of interest. These distances can be increased significantly with the use of larger metallic objects, stronger magnets, etc. Moreover, since magnetic fields 122 are generally unaffected by the presence of water, mud, sand, debris 114, and the like, passive systems 100 should work well in the field. However, slow changes of the Earth's magnetic field and the effect of other metallic parts (for instance, those moving with flowing water) might affect the magnetic field 122 created by various passive sensors 106B. However, passive systems 100 can be constructed to compensate for these and other environmental factors without undue experimentation. Moreover, where it is desired to receive stronger signals from a sensor 106, or group thereof, the receiver (and/or the associated magnetometers, antenna 116 or antennas 116, etc.) can be placed near the region 108 of interest (for instance, an area of a riverbed). In some embodiments, the receiver and/or one or more antennas 116 could be located under water near a submerged portion of the bridge foundation and made to be at least partially water-resistant.

The size, shape, and magnetic strength of the magnets in passive systems 100 can be optimized and calibrated in field applications on bridges and on other structures. Passive systems 100 can be used where measuring the location of a group of sensors 106 and a relatively simple system are desired. However, as disclosed herein, passive systems 100 can be used where measuring the location of individual sensors 106 is desired and/or in more complex situations.

Real-Time Monitoring with Active Sensor Positioning Methods

Several methods of measuring scour using active sensors 106A employing magneto-inductive communication are also disclosed herein. In some active embodiments, active sensors 106A include resonators 128 (see FIG. 6) instead of passive magnets in their housings. These resonators 128 can include devices and/or circuits capable of back scattering (or reflecting) an external magnetic or electromagnetic signal back at least in part in the direction from which the signal came. Such active sensors 106A can be used to measure the position of these active sensors 106A individually or as a group. More specifically, if it is desired to track the location of individual active sensors 106A, then the resonant frequencies of the resonators 128 can be selected so that each active sensor 106A has a resonator 128 with a frequency different from the others. Thus, each of the resonators 128 can be detected and tracked individually in some embodiments. Another embodiment uses passive back-scattering of an externally applied magnetic field to detect the movement of active sensors 106A. More specifically, the sensors 106 can contain a resonating circuit such that they communicate via passively back-scattering a magnetic field or electromagnetic field. In yet another embodiment, the sensors include a component(s) which, when the sensors 106 rotate, disables or destroys the resonator 128 (or magnet). For instance, the component could open the resonator 128 circuit or connect a magnetic short circuit path to the resonator 128 so that the external field decreases noticeable. For instance, the magnetic field could be reduced to a point that it effectively becomes undetectable. Such sensors 106 could be dropped into place and allowed to align themselves with the vertical (for instance). Gap-filler material in the sensors 106 could then be allowed to cure thereby leaving the sensors 106 in place and aligned. If sufficient scour occurs, some or all of the sensors 106 will rotate thereby disabling the magnet 124 or resonator 128 and causing a detectable change in the magnetic field 122 of the sensors 106. As a result, once the sensors 106 move or are re-orientated by more than some selected amount they no longer obscure the magnetic fields 122 of other sensors 106 (such as sensors 106 that might have been added after the sensors 106 that was moved or re-oriented). Again, this change in the magnetic field 122 can be detected and used to determine the corresponding change(s) in scour-related conditions.

Some active embodiments involve enabling magneto-inductive communications between active sensors 106A and a receiver. These communications can be used to identify and locate individual active sensors 106A at frequencies less than 10 MHz in some embodiments. However, higher communication frequencies are within the scope of the disclosure. In some embodiments communication methods such as magneto-inductive or sound-based methods can be used to query active sensors 106A regarding various scour-related conditions.

As with the passive sensors 106B disclosed herein, these active sensors 106A can be configured to have densities, shapes, sizes, etc. selected to mimic naturally occurring rocks (or other filler materials 104). Thus, active sensors 106A can be configured to respond to flowing water in a manner similar to that of naturally occurring rocks.

As illustrated in FIG. 9, a data acquisition/analysis system 900 of one embodiment transmits a magnetic signal 922 that is scattered by various active sensors 906A at selected frequencies. The magnetic signals 922 (as measured in magnitude and/or phase) received by a set of receiver antennas 916 (and/or magnetometers) can be used to determine the position of the individual active sensors 906A and, by comparison with their initial as-placed and subsequent locations, to monitor scour. Moreover, signal processing techniques can be applied to recover even weak or conflicting magnetic signals 922 from active sensors 906A. Antennas 916 of relatively larger size can also be used to increase the ability of active systems 900 to receive and analyze these magnetic signals 922.

The system of the current embodiment can use two communication methods although the disclosure is not limited to these communication methods. For one communication method, some sensors 906A use active magneto-inductive communication links and contain a battery and timer and possibly a receiver to wake up the system. Thus, these particular sensors 906 can happen to transmit information at select times (for instance, every hour). However, the active communications of these sensors 906 could occur via ultrasonic or other types of transmitters. For the other communication method of the current embodiment, some sensors 906B use passive magneto-inductive communication via RF (radio frequency) signals. A transmitter (with a signal strength selected to provide communications between the transmitter and the sensors 906) transmits a signal to the sensors 906. These sensors 906 detect the transmitted signal and send it back to the transmitter (or receiver thereof). These sensors 906 can send the signal back to the transmitter by passive scattering, rectification, activation of an active circuit therein, etc. The active circuits of such sensors 906 can be similar to those found in RFID (radio frequency identification) tags.

In another embodiment, each sensor 106 includes a magneto-inductively powered rectifier. In such embodiments each sensor 106 detects an external signal and rectifies it to power a transmitter circuit that sends (on another frequency) a code for identifying that sensor 106. In addition to the code, these sensors 106 of the current embodiment can send information related to their physical orientations as measured by built-in accelerometers. Some of these built-in accelerometers can be configured to detect the Earth's gravitational field and to compare the orientation of the active sensors 106A (in which it is located) to that field. Further, if desired, the magnetic field of the earth can be measured to remove ambiguity in the determination of the system's orientation that might occur if only the gravitational field of the earth is used. The resulting information can be transmitted and used to measure how much these active sensors 106A have moved or otherwise been reoriented.

In yet another embodiment, some or all of the sensors 106 include a battery and a timer. Some of these timers can draw power from the batteries and can trigger transmission bursts at selected times. As a wristwatch can run on a small battery for three years or longer, a sensor 106 with a timer operating on a battery can last for decades. Since each transmission burst can be of relatively short duration (less than 1 second), the integrated energy consumption will be low in spite of the occasionally increased current draw associated with these transmission bursts. Moreover, the underwater environment where such sensors 106 are expected to typically reside in operation is favorable for batteries (temperatures in such locations varies slowly and over a limited range) thus enabling long battery lives. Nonetheless, active sensors 106A of the current embodiment can be configured to minimize the energy consumption from self discharge and from standby circuits to extend the battery life of some active sensors 106A. It is expected that a battery life of 10 years is achievable for at least some active sensors 106A.

Active sensors 106A (see FIG. 1) of various embodiments include mechanisms, circuits, etc. which allow a user to select from a variety of transmission scenarios. Three such embodiments include:
An embodiment in which active sensors 106A are activated by an external magnetic, magneto-inductive, radio frequency, ultrasonic or other type of signal and when activated transmit their IDs, and other information at the same frequency as the eternal signal or at some other frequency;
Another embodiment wherein the active sensors 106A include timers and transmit their ID, and other information regularly, such as hourly; and
Yet another embodiment in which each active sensor 106A is activated by an accelerometer and transmits its ID, and/or other information as the sensor rotates or moves (with a timer determining how long it remains transmitting after its movement or reorientation).

The latter variant can be applied to automatically alert an engineer-in-charge (or other users) to evolving scour-related information through an Internet or telephony connection or other telecommunication techniques. Such active sensors 106A could transmit a variety of information related to scour including the distance to other sensors 106 from itself and/or acoustic noise at its location.

Preliminary simulations regarding the signal-to-noise ratio show the possibility of communications through water and between active sensors 106A and receivers at frequencies below 1 MHz (although communication at higher frequencies is also included in the scope of the disclosure). In a non-optimized scenario, communication between an active sensor 106 at a depth of 2 m in fresh water was achieved with a receiver using two 0.5 m×0.5 m antennas 116. One antenna 116 of this system 100 was placed 5 m to the left and the other antenna 116 was placed 5 m to the right of the sensors 106. Performance of other systems 100 can be improved by using larger antennas 116, more turns, higher power, and/or various signal processing techniques. Alternatively, the antenna(s) 116 may be installed around a bridge pier 110 close to the potential scour region 108 to minimize the communication distance.

As disclosed herein, different types of sensors 106 (with passive structures inside, with semi-active structures inside, and with batteries or other active components inside) are provided in this disclosure. The ability to communicate with those sensors 106, to locate the sensors 106, and the complexity and life spans of the systems 100 can be factors to consider in selecting a system 100 for scour measurement and/or mitigation applications.

The size of the antennas 116, the number of antennas 116 and their position relative to the potential scour region 108 can influence the ability to communicate with and locate various types of sensors 106. If the antennas 116 are embedded into piers 110 (for bridges under construction or those being retrofitted), they can be close to the sensors, thus improving the communication with, and the localization, of such sensors 106.

Signal processing is another way to improve the quality of the information received from the sensors. There are many ways to process signals and extract information even from weak or conflicting signals. Any of these and other techniques can be employed to improve the recovery of the information transmitted from (or otherwise provided by) sensors 106 in a given system 100.

If a magneto-inductive communication is considered, the salinity (and therefore conductivity) of the water can influence the ability to communicate magneto-inductively with, and to locate, sensors 106 deployed in such environments. Typically, lower communications frequencies allow for better communication through salt water. Moreover, systems 100 of some embodiments can use magneto-inductive, sonic, ultrasonic, electromagnetic, and other methods of communicating scour-related information to the receiver. Thus it is possible that different system 100 designs can be optimized for different situations based on tradeoffs between antenna size, antenna positioning, communications frequency(s), power, reliability, etc.

Recently, some electromagnetic simulations were conducted at Missouri S&T to evaluate the feasibility of active sensor positioning. The results indicate that it is possible to reconstruct the paths of individual sensors 106 during the scour process. Doing so can entail integrating the signals from one or more accelerometers on the sensors 106 of interest. In addition, or in the alternative, the overall movement of a particular sensor 106 or groups of sensors 106 can be monitored with active systems 100.

Sensors 106, systems 100, and their wireless sensing networks of various embodiments provide non-limiting advantages over technologies heretofore available. First, the architecture of sensors 106 (such as the magnetic sensors of various embodiments) and their wireless sensing networks can be relatively simple. They can be easy to install and the data acquired there from can be easy to process. One pertinent measurement principle used in such embodiments is based on classical magnetic field theory. In some embodiments an inverse transform is performed to identify the presence of sensors 106 from measured magnetic field data. Systems 100 of many embodiments require minimal (or no) professional services to install and operate in practical applications. Moreover, systems 100 can be installed at the time of foundation construction, when scour monitoring is desired, during scour events, and/or during scour mitigation efforts.

Second, sensors 106 can be durable and applicable to environments with high water velocities, debris 114 or ice entrained in a current. With the protection offered from the bodies or housings 126 of some sensors 106, the embedded devices can survive various harsh environments and can be operational throughout the life span of an engineering structure (for instance, bridges or offshore platforms).

Third, sensors 106 can be multi-functional. More particularly, systems 100 can combine the scour monitoring and scour protection/mitigation into one integrated implementation. Systems 100 of embodiments can be applied to the bottom of the bodies of water formed from soil, rocks, sand, other materials, or various combinations thereof thereby extending the application range of systems 100 beyond that of previously available technologies.

Fourth, systems 100 can be small, portable, and easy to deploy. For instance, a group of sensors 106 and a magnetometer 120 (or other receiver) can be configured to fit in a back pack which can be carried to a place near, or at, a potential scour site. The magnetometer 120 can be deployed and the sensors 106 dropped or otherwise placed in a region 108 of interest. The initial magnetic field 122 and changes thereto can be measured with the magnetometer 120 with data analysis occurring at the same (or some other) time. Nonetheless, the deployment of systems 100 of the current embodiment can take only a few moments or less.

Next, such systems 100 as those disclosed herein can be inexpensive. According to the Hydrologic Engineering Center (HEC), the instrument costs of various scour monitoring technologies are approximately: $2000 for physical probes, $15,000 for a portable sonar survey grade system, $5,000-$15,000 for a fixed sonar, $7,500-10,000 for a sounding rod, $5,000-10,000 for a magnetic sliding collar, $3,000 for a float-out system or $500/float-out, $10,000 for traditional land survey, and $5,000 and $20,000 for Global Position System (GPS) of sub-meter and centimeter accuracy, respectively. See Lagasse, P. F., Richardson, E. V., and Schall, J. D., “Instrumentation for Monitoring Scour at Bridge Piers and Abutments,” NCHRP Report 396, Transportation Research Board, National Research Council, National Academy Press, Washington, D.C., 1997, p. 109. In comparison with these previously available technologies, a system of twenty sensors 106 of some embodiments might cost as little as $1,000.

Thus, embodiments provide solutions to the largest cause of bridge collapses in the United States due at least in part to their ease of use, low or non-existent maintenance considerations, cost effectiveness, and/or other considerations. Moreover, sensors 106 of various system themselves can be used to mitigate scour conditions since they can be configured to have pertinent characteristics (for instance, density) similar to those of naturally occurring rocks and/or other objects sometimes used to mitigate scour-related conditions. Another advantage provided by embodiments is that some systems 100 can be placed on and/or near existing bridges whereas previously available systems 100 and/or methods need special installations on the bridge and/or under the water.

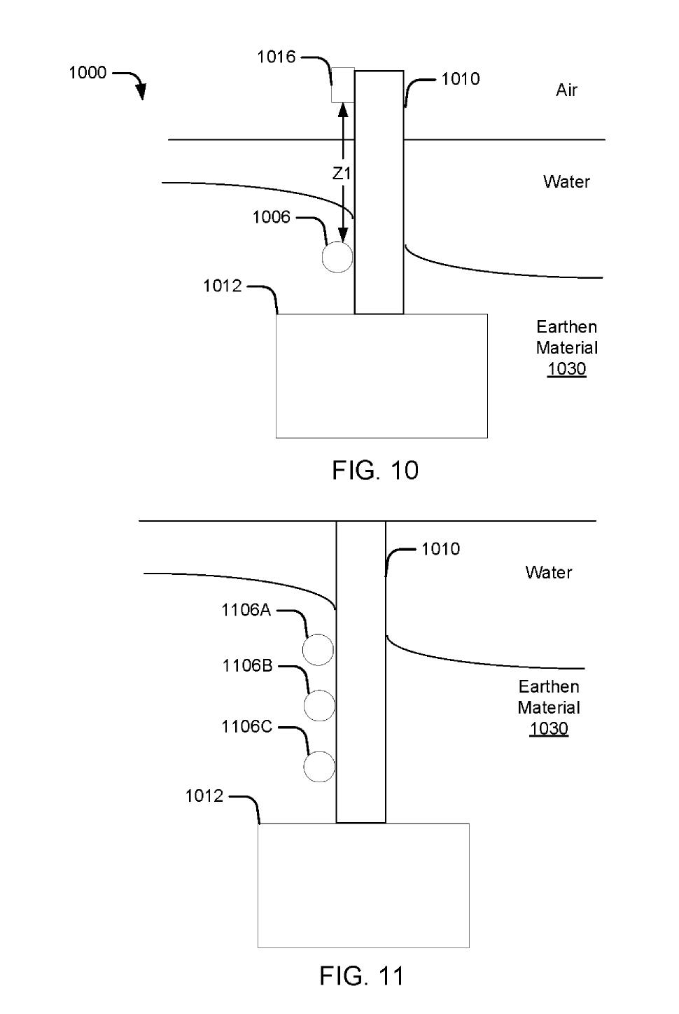

FIG. 10 illustrates an erosion measurement system. More particularly, FIG. 10 illustrates that a structure 1010 and/or 1012 which might be subject to the affects of erosion has been instrumented to detect that erosion and to measure its extent in a uniaxial manner. In other words, the sensor 1006 and receiver 1016 are aligned with one another so that the location of the sensor 1006 relative to the receiver 1016 can be readily determined without triangulation or other more computationally demanding methods. This result is so because the sensor 1006 is mechanically constrained to travel in only one dimension (here vertically). In some case, though, the sensors 1006 can be configured to possess high enough densities that they are likely to move in only one direction (for instance, vertically) even if not mechanically constrained. Thus, given the received signal strength (which correlates with distance from the receiver 1016), the location of the sensor 1006 can be determined without having to align the antennas with each other. In other words, if the sensor's orientation is known, its depth will follow more or less directly from the received signal strength.

In some cases the erosion could be scour or other types of erosion such as the displacement caused by a landslide (wherein the structure might be a retaining wall, highway, tunnel opening, etc.). Moreover, the structure (and its environs) has been instrumented with an erosion monitoring system 1000 which happens to include a receiver 1016 and one or more sensors 1006 (either active, passive, or a combination thereof). In the current embodiment, the sensor 1006 was buried in the earthen material 1030 (soil, sand, gravel, stones, sediment, clay, regolith, and/or the like.) during the construction, repair, or retrofit of the structure 1010. More specifically, the sensor 1006 was placed directly under the receiver 1016 at some generally vertical distance z1 therefrom. Prior to its placement, the properties of the sensor 1006 were chosen so that the sensor 1006 happens to be heavier than the earthen material 1030, the water, and other materials likely to be present in the environs of the instrumented structure.

As a result, as earthen material 1030 erodes from under the sensor 1006, the sensor 1006 will sink in a generally vertical fashion. Since the magnitude of the signal received by the receiver 1016 from the sensor 1006 depends on the distance between the two devices, the signal strength provides a more or less direct measurement of the distance z1 and, hence, the extent of the erosion present at the location of the sensor 1006. The receiver 1016 can be queried to obtain the distance z1 (or signal strength) by any available method such as by querying it with a magneto-inductive signal or an acoustic signal. The former being useful when the receiver 1016 is located in the air and the latter being useful when the receiver is located in water. Although, other signaling schemes could be employed.

FIG. 11 illustrates another erosion measurement system. For the embodiment of FIG. 11, another uniaxial system 1100 includes multiple sensors 1106 buried at differing depths in the earthen material 1030. For instance, a borehole could be drilled alongside an existing structure and the sensors 1106A, 1106B and 1106C could be placed therein at differing depths. As erosion subsequently occurs each of the sensors 1106 will therefore likely be carried off in turn.

Thus, each sensor 1106 will at some time likely become exposed to the forces causing the erosion and roll or tilt accordingly as the erosion reaches its depth. As a result, sensors 1106 equipped with tilt/roll/yaw sensor(s) and placed in accordance with FIG. 11 could indicate the onset of erosion at their corresponding depths when they detect some change in their orientation. Moreover, such erosion sensing could occur before the sensor 1106 involved has sunk appreciably since the forces of erosion might reorient the sensor 1106 well before the sensor 1106 begins sinking. In other words, in some situations, the onset of erosion at the depth at which the sensor 1106 involved was placed when the orientation of the sensor 1106 changes. Moreover, the association of the onset of erosion can be identified with the location (in the other two dimensions) of the identified sensor without triangulating or otherwise determining the location of the sensor (other than perhaps recording the location of the sensor when it was placed).

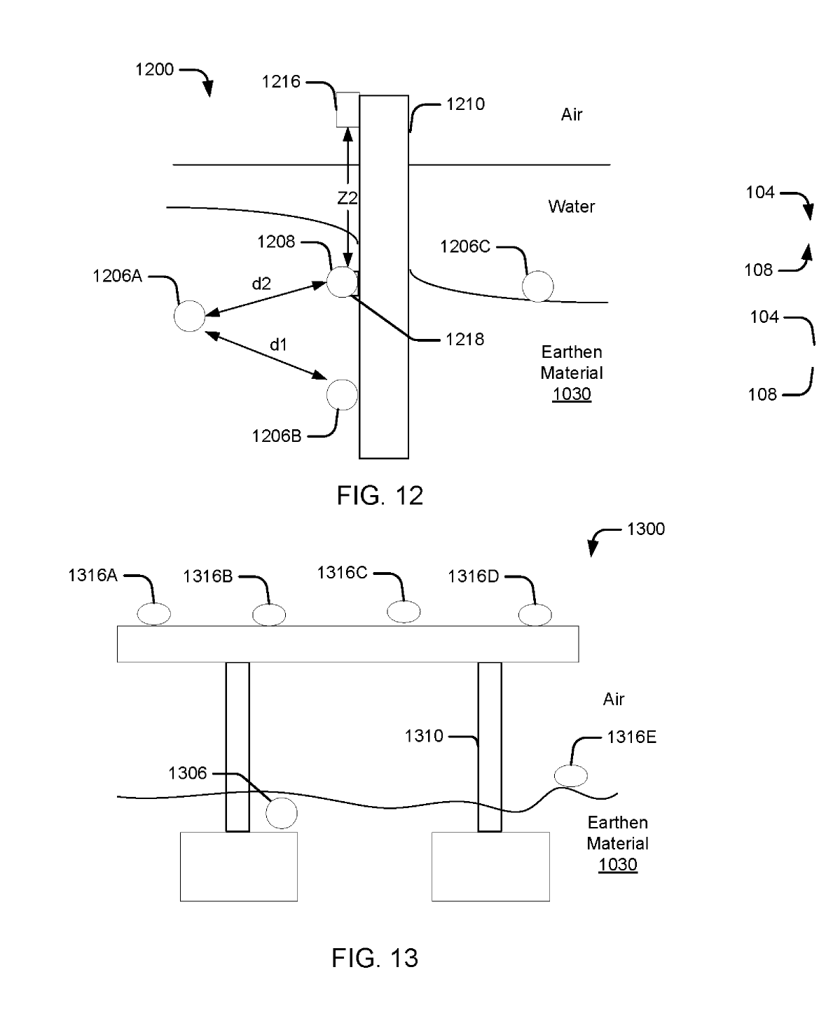

FIG. 12 illustrates yet another erosion measurement system. More specifically, FIG. 12 illustrates a system 1200 of an embodiment which includes an inter-sensor network configured to measure the distances d1 between various sensors as well as measure the extent of erosion as sensed by those sensors. For instance, the system 1200 can measure the effects of erosion by way of measuring the relative locations of the sensors 1206. System 1200 can be used at locations where the sensors 1206 will be deployed at greater distances from the network device 1216 (or master sensor) and/or a structure 1210 on which the network device 1216 might be mounted. For instance, a network device 1216 might be mounted on or near the deck of a tall bridge (to facilitate access). In that case, the distance z2 down to the sensors 1206 from the network device 1216 might be on the order of 100 or more feet. In contrast, the sensors 1206 could be located in closer proximity to each other than to the network device 1216. Thus, knowing the antenna radiation patterns and the derived path losses, a system of equations can be set up to determine the location of the sensors 1206 relative to each other and/or relative to a fixed location (such as the location of the base station 1208).

As FIG. 12 illustrates, the system 1200 includes numerous sensors 1206A-C, a network device 1216, and a master sensor or base station 1208. If desired, the base station 1208 can include a relatively large antenna, more processing capacity, etc. than the other sensors 1206 (which might be relatively simple or even passive sensors). For instance, the base station 1208 could include multiple antennas in case some of the sensors 1206 are shadowed by structure, debris, etc. As is disclosed further herein, the base station 1208 can measure received signal strengths from the other sensors 1206; can have a fixed location; and can remotely power the other sensors 1206 through magneto-inductive coupling (for instance). Of course, various sensors can have an internal battery and/or an energy harvesting circuit. Moreover, since the base station 1208 usually happens to be closer to the other sensors 1206 than the network device 1216, it will typically receive stronger signals from the other sensors 1206 than the network device 1216 would (or does).

Each of the sensors 1206 includes an internal receiver with which it listens for signals (be they magneto-inductive, sonic, etc.) from the other sensors 1206 and measures the received strength thereof. Since the sensors 1206 can be configured to transmit their identifiers in the signals, each of the sensors 1206A can identify the other sensors 1206B and the distances d1 thereto (by correlating the received signal strengths with those distances d1). Furthermore, each sensor 1206A can transmit a signal to the base station 1208 conveying its identifier, and the identifier/distance pairs associated with the other sensors as measured by it. The sensors 1206 of the current embodiment also transmit their own identifier and the identifier/received-signal-strength pairs. Of course, for each signal received, each sensor 1206 can associate a distance d1 (or received signal strength) with the identifier conveyed by the signal thereby forming another identifier/distance pair.

For its part, the base station 1208 can be configured to receive the signals from the sensors 1206 conveying the identifier/distance pairs which they developed. Moreover, the base station 1208 can re-transmit this information to the network device 1216. The base station 1208 (or network device 1216) can use this information to locate the sensors 1206 which are in communication with each other and/or the base station 1208. In other words, the base station 1208 can use the collection of identifier/distance pairs to form and solve a system of simultaneous equations for the locations of the sensors 1206 (relative to the base station 1208). These systems of equations can make use of a priori knowledge of the antenna patterns and/or transmission strengths of the sensors 106. Note also, that the base station 1208 can measure the received signal strengths of the signals reaching it from each of the sensors 1206 to provide another set of identifier/distance d2 pairs associated with the sensors 1206 which it can sense. Here, distance d2 happens to be the distance between a sensor 1206 and the base station 1208. In addition, note that some of the sensors 1206 can be too far from the base station 1208 to effectively communicate with it. However, these relatively distant sensors 1206 can still participate in the system 1200 by communicating indirectly through another of the sensors 1206 acting as an intermediary in the network.

Note that the base station 1208 can be configured to remain in a fixed location even during erosion inducing events such as landslides, floods, etc. For instance, the system 1200 can include a bracket 1218 attached to the base station 1208 and coupled to the structure 1210. In the alternative, or in addition, the base stations 1208 can be dense or heavy enough that, given expected conditions, the erosion inducing event(s) would be unlikely to move it. FIG. 12 also illustrates that the base station 1208 can communicate with the network device 1216. The signals between the base station 1208 and the network device 1216 can take a variety of forms. For instance, the signals between the base station 1208 and the network device 1216 can be magneto-inductive, acoustic, etc.

Another aspect of system 1200 is that it allows users to calibrate network devices 1216 for local/instantaneous conditions. In other words, upon setting up a new network device 1216 (or at other times) the user can measure the received signal strength from the base station 1208 using the network device 1216 (or a magnetometer) and, given the known distance z2, calibrate the network device 1216 for current conditions.

System 1200 can include only one network device 1216 thereby simplifying the “on-bridge” (or other structure) portion of the system 1200. More specifically, if a portable system 1200 is sought, a one-network-device system 1200 can be used since the single network device 1216, base station 1208, and collection of sensors 1206 can be carried more easily than the corresponding components of a multi-receiver system.

In some embodiments most, if not all of the processing is performed in the network device 1216. In other words, the sensors receive signals from each other, determine the received signal strengths and pair that information with the corresponding sensor identifiers. The sensors 1206 transmit the received signal strength/identifier pairs to the base station 1208. The base station 1208 determines the received signal strengths from each of the other sensors 1206 and appends the resulting information to the other received signal strength/identifier pairs. The base station 1208 forwards the compiled information to the network device 1216 which then solves for the location of the sensors 1206.

Moreover, it might be worth noting that system 1200 acts much like an ad hoc network with the sensors 1206 communicating among themselves and/or with the base station 1208. Thus, even if a particular sensor(s) 1206 malfunctions, the system 1200 will continue to operate with, at worst, one set of identifier/distance pairs missing.

FIG. 13 illustrates still another erosion measurement system. The system 1300 of the current embodiment includes at least one sensor 1306 and multiple network devices 1316 and (while not shown) a processor in communication with the network devices 1316. The processor or some other device can localize the sensor(s) 1306 by triangulating the signals received from that particular sensor 1306 (as received by the network devices 1316A-E, base stations, and/or other receivers). Thus, the network devices 1316 can be placed in or near the region in which erosion is expected and their locations can be recorded. The sensor 1306 can then be placed in the region of expected erosion and activated (if/when desired). Whether the sensor 1306 is active or passive, each of the network devices 1316 can receive one or more of the signals which it generates and measure the signal's received signal strength. The distances between the various network devices 1316 and the sensor 1306 can then be determined by triangulation techniques.

Of course, as erosion moves the sensor 1306, the new or changing location of the sensor 1306 can be obtained by triangulation. By selecting the locations of the network devices 1316 relative to the location (and/or expected location) of the sensor 1306 to provide resolution in all three dimensions, the location of the sensor 1306 can be determined in all three dimensions. It is noted here that the improved spatial resolution available with increasing inter-receiver spacing can be balanced against the greater signal strength at decreasing sensor-to-receiver distances. Indeed, systems 1300 of some embodiments include 4 or more network devices 1316 with at least one being vertically offset from the others. Moreover, at least one network device 1316 can be place on either side of the sensor 1306 as seen looking along the direction in which the sensor 1306 is expected to move. Furthermore, some network devices 1316 can be placed upstream and some downstream from the location and/or expected location(s) of the sensor 1306. For instance, one or more network devices 1316 can be placed on a structure 1310 for which erosion data is sought.

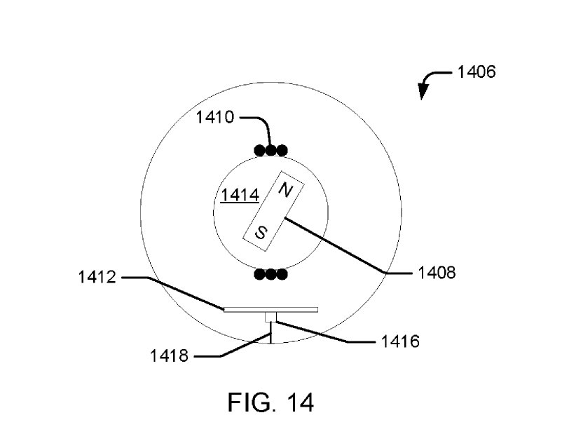

FIG. 14 schematically illustrates an erosion sensor. The erosion sensor 1406 of the current embodiment allows an erosion measurement system to more easily identify particular sensors 1406. In part it does so by reversing or “flipping” the magnetic field generated (or associated with) the sensor 1406. Thus the sensor includes a magnet 1408, an electromagnetic coil 1410 and a printed circuit board (PCB) 1412 and defines a chamber 1414. The magnet 1408 is mounted in the housing of the sensor in such away that it can rotate about at least one axis in the chamber 1414. For instance, the coil 1410 could be mounted on an axle running through the chamber 1414. In the current embodiment though, the magnet 1408 floats in (or rests in) oil or another liquid which fills the chamber 1414.

FIG. 14 also illustrates that the coil 1410 is positioned relative to the magnet 1408 so that when it is energized the resulting electromagnetic field causes the magnet 1408 to reorient itself. For instance, the coil 1410 could be wrapped around a portion of the sensor 1406 which defines the chamber 1414. Moreover, the coil could be powered by circuitry on the PCB 1412 which would control when/if the coil 1410 energizes. A receiver could also command the sensor 1406 to reverse (or at least change) the magnetic field associated with the magnet 1408. The PCB 1412 could then energize the coil 1410 to cause the magnet 1408 to reorient itself accordingly. Then, some time later, the PCB 1412 could reverse the current through the coil 1410 causing the magnet 1408 to rotate or flip (this time completely reversing its magnetic field if desired). Of course, this process could repeat as often and/or as frequently as desired. In any case, with the magnetic field reversing (in whole or in part), a receiver could more readily identify and locate the sensor 1406 thereby improving system level performance. Note also that by reversing the magnetic field of the magnet 1408, the (relatively constant) environmental field can be isolated and accounted for during the determination of the location of the sensor 1406.

It might be worth noting at this juncture that both the Earth's (or environmental) magnetic field and the coil's magnetic field (when energized) orient the magnet 1408 of the current embodiment. Thus, if the environmental magnetic field is known or can be measured, then the field coupling pattern of the magnet 1408 can be determined mathematically thereby facilitating detection of the sensor 1406 and determination of its orientation and location (using either magnitude or phase techniques or a combination thereof).

Moreover, if desired, various sensors 1406 could include pressure sensors 1416 to aid in determining the depth at which the sensors resides. Thus, when the sensors 1406 are placed in water the water pressures sensed by the pressure sensors 1416 give an indication of the depths of the sensors 1406. Sensors 1406 of such embodiments can find use in water and/or in water-saturated earthen materials. Moreover, some sensors 1406 couple the pressure sensor 1416 to a sealed tube 1418 which has a flexible diaphragm at the end flush with the surface of the sensor 1406. Thus, the pressure sensor 1416 communicates with the ambient environment while being protected from the environment (particularly water) and need not itself be waterproof. Moreover, the rest of the sensor 1406 can be sealed and/or filled with some inert liquid to make the sensor 1406 more waterproof than might otherwise be the case.

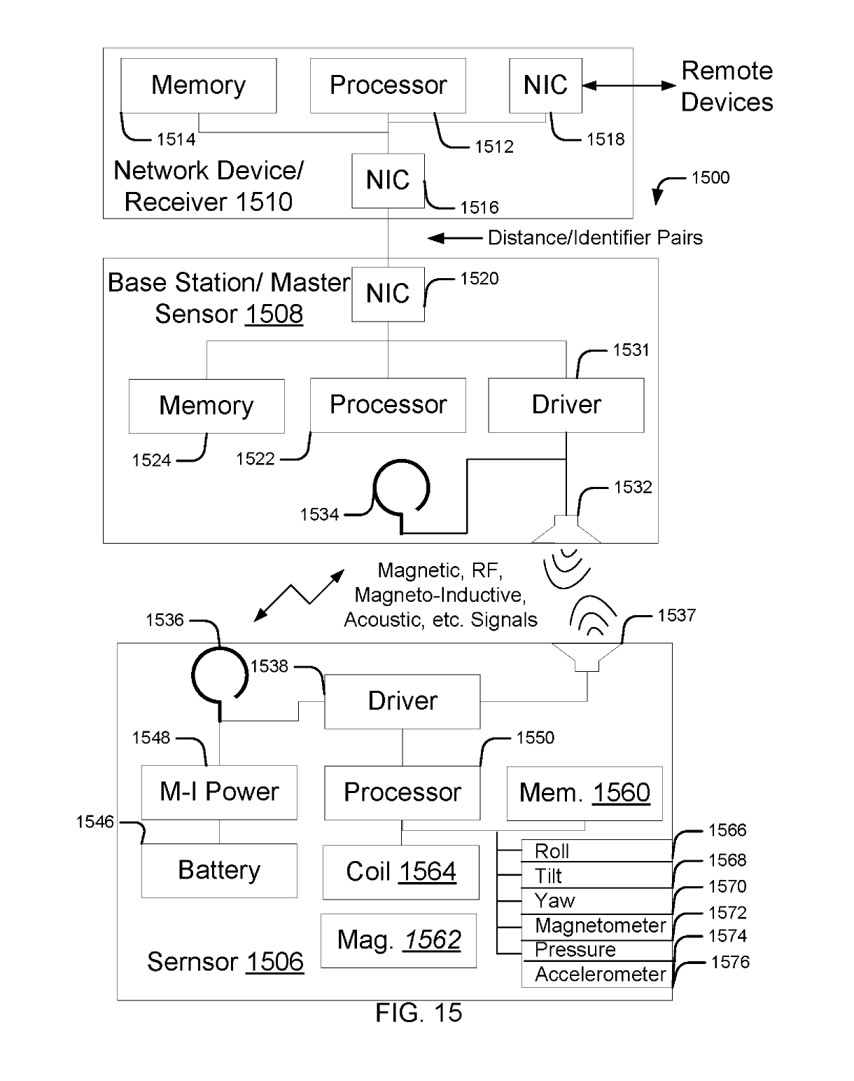

FIG. 15 illustrates a block diagram of an erosion measurement system. More specifically, FIG. 15 illustrates system 1500 in which various sensors 1506 form a network together with a base station 1508, a network device 1510, and (perhaps) other devices located remotely from the rest of the system 1500. Generally, the sensors 1506 communicate among each other thereby forming a network and sensing the received signal strengths of the signals from each other. From the received signal strengths a particular sensor 1506 can determine how far away the other sensors 1506 are from itself. Moreover, each sensor can have an identifier which it transmits with its signal thereby allowing the other sensors 1506 to form sensor identifier/distance (or received signal strength) pairs. Some or all of the sensors 1506 can also each broadcast a signal which conveys the identifier/distance pairs which that particular sensor 1506 has developed. In addition, or in the alternative, the base station 1508 could listen for and receive such identifier/distance pairs. From the accumulation of identifier/distance pairs, the base station 1508 can determine the relative locations of the sensors 1506 in the system 1500. The resulting information can be stored at the base station 1508 and/or can be transmitted to the network device 1510 for further distribution if desired. In this sense, the network device 1510 could serve as a data link to a telecommunications system such as a cellular telephony system, a satellite-based communication system, a wireless network, etc.

With continued reference to FIG. 15, the network device 1510 can include a processor 1512, a memory 1514, and a pair of network interface cards 1516 and 1518 (or other form of communication circuitry). In the current embodiment, the network device 1510 communicates electronically with the base station 1508 and with other devices on a network via respectively NICs 1516 and 1518. In the current embodiment, the processor 1512 performs various algorithms for determining the relative and/or absolute locations of the sensors 1506 from the identifier/sensor pairs and its knowledge of the location of the master sensor 1510. It can store the results thereof in the memory 1514 which can also serve to store the various algorithms, programs, code, etc. for doing so and/or for operating the network device 1510. The NICs 1516 and 1518 serve to transform communications to/from the processor 1512 to forms suitable for the communication methods employed by the network device 1510 and in accordance with various protocols, standards, etc.

With continuing reference to FIG. 15, the base station 1508 of the current embodiment includes many components similar to those discussed with reference to the network device 1510. Thus, the base station 1508 also includes a NIC 1520, a processor 1522, and a memory 1524. In addition, the base station 1508 includes several circuits including a driver circuit that can include several sub-circuits including a modulator, a demodulator, a mixer, an amplifier, etc. For the sake of convenience the various combinations will be referred to as a driver 1531. In addition, the base station 1508 of the current embodiment includes a microphone/speaker or other acoustic transducer 1532 and a loop antenna 1534.

Thus, when it is desired for the base station 1508 to either power a sensor 1506 via a magneto-inductive signal or to communicate with a sensor(s) 1506, the processor 1522 sends a signal to the driver 1531 indicative of which output device to use. Depending on whether the processor 1522 has selected the acoustic transducer 1532 or the antenna 1534, or both, the driver 1531 routes the modulated signal to the selected output device. Note that it has been found that for both acoustic and magneto-inductive transmissions the driver 1531 can drive the selected output device(s) at the same frequency. In some embodiments, the driving frequency is 125 kHz+/−5 kHz. In other embodiments, the driving frequency is 250 kHz. At or near these frequencies both communication methods work sufficiently well for many applications. However, it is expected that frequencies as low as 5 kHz can be used with success in certain situations. The selected output device 1532 or 1534, of course, drives the modulated signal out into the environment.

Still with reference to FIG. 15, the sensors 1506 of the current embodiment can also include a variety of components. For instance, they include either an antenna 1536 an acoustic transducer 1537, or both. Additionally, the current embodiment provides a transceiving circuit or driver 1538 in the sensor 1506. Like the driver 1531 of the base station 1508, the driver 1538 of the sensor 1506 can perform a variety of functions including modulation, demodulation, mixing, amplification, etc. as appropriate for the type(s) of input/output devices 1536 and/or 1537 on the sensor 1506. The sensors 1506 can include a battery 1546 for powering the various components of the sensor 1506. In addition, or in the alternative, the sensors 1506 can include a magneto-inductive power harvesting circuit 1548 coupled to the antenna 1536 for deriving power from magneto-inductive signals received by the antenna 1536. Either or both sources of power can be used to power the sensor 1506.

Additionally, the sensors 1506 of the current embodiment can include a processor 1550 and a memory 1560. The processor 1550 can execute various programs, codes, algorithms, etc. for operating the sensor 1506. Of course, the memory 1560 can provide storage capabilities for such algorithms, codes, programs, etc. and for data generated or used in the course of sensor 1506 operations.

FIG. 15 also illustrates that sensors 1506 of the current embodiment include a magnet 1562 and an associated coil 1564. As disclosed elsewhere herein, the coil 1564 and magnet 1562 can be positioned relative to one another such that the coil 1564 can rotate the magnet 1562 and thereby controllably reverse or alter its orientation. Moreover, because the processor 1550 is connected to the coil 1564, the processor 1550 can control this process as desired.

In addition, sensor 1506 of the current embodiment includes a variety of transducers. For instance, sensor 1506 can include roll, tilt, and yaw sensors 1566, 1568, and 1570 respectively. In the alternative, or in addition, the sensor 150 can include an accelerometer 1576 or other device to sense acceleration (or movement) along one or more translational or rotational axes. The sensors can also include a magnetometer and/or pressure sensor 1572 and/or 1574 as desired. Note that integrating the roll, tilt, yaw, and acceleration signals from sensors 1566, 1568, 1570, and 1576 allows one to reconstruct the path that a sensor has taken through floodwaters. Indeed, the feasibility of this capability has been demonstrated by Missouri S&T using computer simulations.

A prototypical system 1500 was recently constructed at Missouri S&T. In the prototypical system a Microchip PIC16LF1823 (available from Microchip Technology Inc. of Chandler, Ariz.) served as the processor 1550 of the sensor 1506. In part, it was chosen for its low power abilities. Communications between the various components of a prototype sensor were implemented using a Phillips I2C compatible bus. Moreover, the driver 1538 was implemented using the EUSART (Universal Asynchronous Receiver Transmitter) capabilities of the processor described above. Successful sensor-to-receiver communication tests were conducted at both 125 and 250 kHz in air, fresh water, and salt water using a loop antenna network device 1510 which measured less than a meter in diameter for the network device 1510.

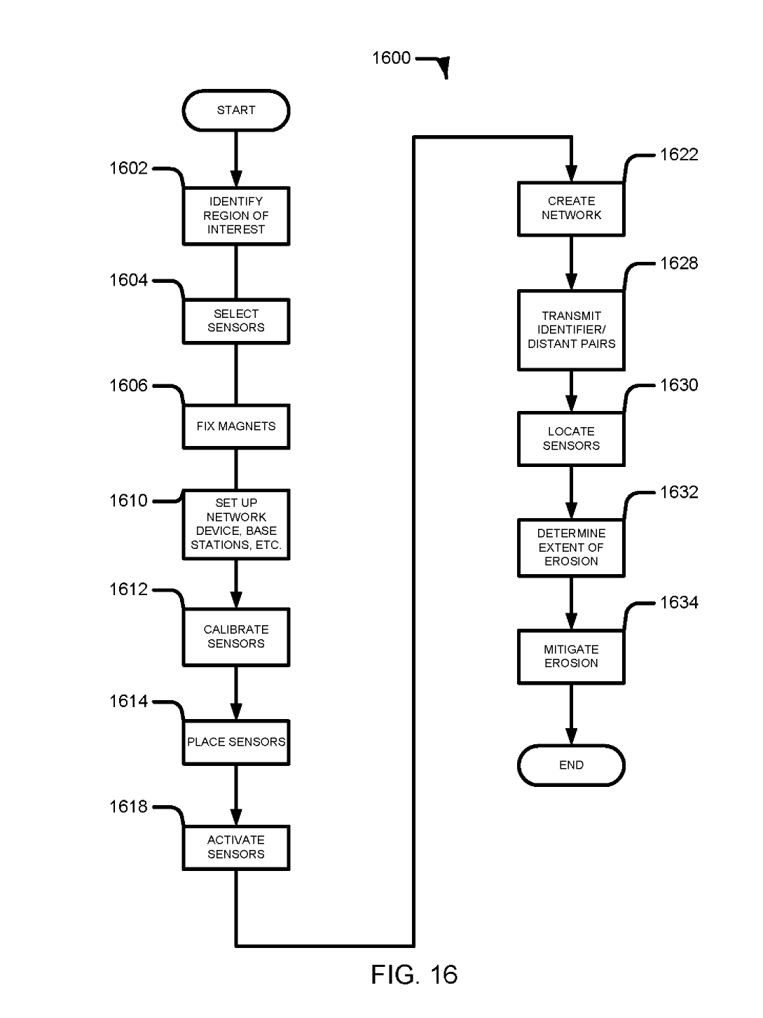

With reference now to FIG. 16, the drawing illustrates a flowchart of a method for measuring erosion. More specifically, FIG. 16 illustrates method 1600 which can include identifying a region in which erosion might occur. For instance, a hill with slumping soil, a bridge over a flood prone river, etc. might be selected. See reference 1602.

At reference 1604 various sensors 106 (see FIG. 6) and their corresponding receivers can be selected. For instance, a group of active sensors 106A or a group of passive sensors 106B can be selected for use in the region identified at reference 1602. In the alternative, a mixture of active and passive sensors 106A and 106B can be selected for use in the region. In some instances though it might be deemed sufficient to use one or a limited number of sensors.

If passive sensors 106B (are to be used and if it is desired to fix the orientation of the magnets therein), then at reference 1606 that can be accomplished. For instance, the magnets can be placed in the sensors and an RTV (room temperature vulcanizing material or some other curable material) can be poured in around them. This action will allow the magnets to orient themselves in a vertical direction if desired. The RTV can then be cured thereby fixing the orientation of the magnets in the sensors 106B.

The receivers can also be set up near the region where the sensors 106 are to be placed as illustrated at reference 1610. Of course the sensors 106 could be placed first. But often, the receivers will be set up first for reasons which will become clear. For instance, with the receivers set up, the sensors 106 can be calibrated one at a time (or in groups) while they are more readily accessible to the users. In some situations, one sensor 106 will be brought near the receiver and the receiver's response noted. Then, the sensor 106 can be moved away from the receiver by some select distance and the response again noted. Thus, it will be known how the sensor 106 and receiver combination respond to changes in their relative locations. The process can be repeated for each of the sensor 106 and receiver combinations. In addition, or in the alternative, a group of sensors 106 (particularly passive sensors 106B) can be calibrated by moving the sensors 106 away from/toward the receiver. Moreover, should it be desired, the passive sensors 106B can be selectively re-oriented and/or scattered to emulate the randomization that might occur during erosion. See reference 1612.

At some point, the sensors 106 can be placed in the region of interest. For instance, if a new structure is being built, the sensors 106 can be buried near the structure, left on the surface, or pre-positioned elsewhere at select locations. More particularly, sensors 106 can be placed away from the structure and/or in the region as desired. The locations of the sensors 106 can be recorded (by, for instance, photographing the scene) if desired. See reference 1614. Of course, sensors 106 could be placed near or retrofitted onto existing structures.

At reference 1618 some or all of the sensors can be activated. For instance, magneto-inductive power can be broadcast to the sensors 106 by the receiver to activate/power them. In the alternative, or in addition, an activation signal can be sent to those sensors 106 configured to remain in a low power state until receiving a signal indicating that some other power state is desired. As a result, various sensors 106 will become active (for instance, some passive sensors 106B might begin back scattering the magneto-inductive signals generated by the receiver). Of course, some active sensors 106A can be activated before they are placed in the environment.

If sensors 1506 which are capable of forming a network among themselves have been activated, it is possible that they will begin forming a network at about this time. For instance, a particular sensor 1506 might recognize a signal coming from a receiver and establish communications therewith. See reference 1622. Moreover, another sensor 1506 might begin broadcasting identifier/distance pairs which the other sensors 1506 then begin to receive.

As the system continues to operate, the various sensors 1506 can continue transmitting identifier/distance pairs as illustrated by reference 1628. Of course, the sensors 1506 can continue to transmit the other readings which they are configured to gather. For instance, indications of their roll, tilt, yaw, acceleration, etc. can be transmitted along with (or separately from) the identifier/distance pairs.

The base station 1508 (and/or network device 1510) can gather the various identifier/distance pairs (and other information). At some time, the base station 1508 can assemble the identifier/distance information into a list and transmit that list to the network device 1510. From that information, the network device 1510 can determine the locations of the various sensors 106 by solving a set of simultaneous equations which models the locations of the sensors 106. See reference 1630. Moreover, having located the sensors 106, the network device 1510 can determine how much erosion has occurred as illustrated at reference 1632. As a result, users can mitigate the erosion if desired by, for instance, placing filler material and or sensors 106 in the region. See reference 1634.

Thus, a number of embodiments have been provided for measuring erosion and related phenomenon such as scour. For instance, some embodiments provide passive DC magnetic techniques and technologies wherein measurements are performed by measuring the generally DC field in the vicinity of a group of sensors.

Other embodiments provide techniques and technologies wherein the magnets of the sensors are aligned with each other (sometimes vertically) and then fixed in orientation. In such cases the orientations of the magnets can be fixed by a process involving the curing of an RTV, glue, or other material. The sensors are then placed with the orientations of the as-placed magnets in alignment with each other. If conditions re-orient the sensors the alignment of the group of magnets will be lessened and the total magnetic field associated with the group of the sensors will decrease accordingly. Thus, changes in the magnetic field of such systems can be correlated with the extent of erosion in the vicinity of the sensors.

Some embodiments provide active DC magnetic sensors within which the magnets can be remotely re-oriented to create a measureable change in their magnetic fields (relative to the environmental magnetic field) when desired. Moreover, in some embodiments, the sensors include magneto-inductive circuitry for receiving power from an external source.

Still other embodiments provide active sensors with tilt sensors, roll sensors, accelerometers, pressure sensors, and sensors for detecting conditions related to erosion (such as the movement or position and/or orientation of the sensor itself). Some sensors include a magnetometer for sensing (and reporting) the magnetic field in the vicinity of the sensors. Sensors of various embodiments can also be configured to sense and report various internal conditions such as their battery status.

In addition, or in the alternative, some embodiments provide sensors which can communicate with one another and can form a network amongst themselves. Sensors of the current embodiment measure the received signal strength of the signals from the other sensors and (in cooperation with a master sensor, receiver, etc.) determine the relative locations of the sensors in the network.

### CONCLUSION

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A method of measuring erosion comprising:
placing a first sensor in a region through which earthen material is expected to move wherein the first sensor has associated therewith a first sensor identifier;
sensing a condition related to the movement of the earthen material with the first sensor;
receiving, with the first sensor, a first wireless signal conveying a sensor identifier associated with a second sensor;
generating a second signal conveying the first sensor identifier and the second sensor identifier; and
locating the first sensor using the second signal.

2. The method of claim 1 wherein the second signal is a wireless signal.

3. The method of claim 1 further comprising locating the second sensor using the second signal.

4. The method of claim 3 wherein the locating of the second sensor further comprises using an identifier/distance pair associated with the second and first sensors conveyed by the second signal.

5. The method of claim 1 further comprising determining a received signal strength of the first wireless signal using the first sensor.

6. The method of claim 1 further wherein the second signal further conveys an indication of the received signal strength of the first wireless signal.

7. The method of claim 1 wherein the sensed condition related to the movement of the earthen material is a position of the first sensor.

8. The method of claim 1 wherein the sensor identifiers are based on frequencies associated with the respective sensors.

9. A system for measuring erosion comprising:
a first sensor adapted to be placed in a region through which earthen material is expected to move and to sense a condition related to the movement of the earthen material;
a first sensor identification being associated with the first sensor;
the first sensor further comprising a first receiver configured to receive a first signal, the first signal being a wireless signal, the first wireless signal to convey a second sensor identification associated with a second sensor; and
the first sensor further comprising a signal generator configured to generate a second signal conveying the first sensor identification and the second sensor identification.

10. The system of claim 9 wherein the sensed condition related to the movement of the earthen material is a position of the first sensor.

11. The system of claim 9 further comprising the second sensor.

12. The system of claim 9 further comprising a bracket coupled to the first sensor and being adapted to mount the first sensor to a structure.

13. The system of claim 9 further comprising a second receiver placed outside of the region through which the earthen material is expected to move.

14. The system of claim 9 wherein the first sensor is further configured to determine a received signal strength of the first wireless signal.

15. The system of claim 14 wherein the signal generator is further configured to generate the second signal further conveying an indication of the received signal strength of the first wireless signal.

16. The system of claim 9 further comprising the second sensor wherein the first sensor is mounted on a structure in the region where the earthen material is expected to move and wherein the second sensor is placed in the earthen material.

17. The system of claim 9 wherein the signal generator is further configured to generate the second signal as a wireless signal.

18. The system of claim 9 wherein the first sensor further comprises an accelerometer.

19. The system of claim 9 wherein the first wireless signal is selected from the group consisting of an acoustic signal and a magneto-inductive signal.

20. A system for measuring erosion comprising:
a first sensor adapted to be placed in a region through which earthen material is expected to move and to sense a condition related to the movement of the earthen material, a first sensor identification being associated with the first sensor;
a second sensor having a second sensor identifier associated therewith;
the first sensor further comprising a first receiver configured to receive a first signal from a second sensor, the first signal being a wireless signal, the first wireless signal to convey the second sensor identifier associated with the second sensor; and
the first sensor further comprising a signal generator configured to generate a second signal conveying the first sensor identifier and the second sensor identifier.
